(19) United States
(12) Patent Application Publication
(10) Pub. No.: US 2019/0254638 A1
(43) Pub. Date: Aug. 22, 2019

(54) INFORMATION-PROCESSING APPARATUS, METHOD FOR PROCESSING INFORMATION, INFORMATION-PROCESSING SYSTEM, AND NON-TRANSITORY COMPUTER-READABLE MEDIUM

(71) Applicant: CANON KABUSHIKI KAISHA, Tokyo (JP)

(72) Inventor: Taku Inoue, Machida-shi (JP)

(21) Appl. No.: 16/398,959

(22) Filed: Apr. 30, 2019

Related U.S. Application Data
(63) Continuation of application No. PCT/JP2017/041405, filed on Nov. 17, 2017.

(30) Foreign Application Priority Data
Nov. 24, 2016 (JP) 2016-228064
Oct. 16, 2017 (JP) 2017-200400

(51) Int. Cl. A61B 8/08; G16H 30/20; G16H 40/63; A61B 5/00; A61B 8/00

(52) U.S. Cl. CPC A61B 8/5261; G16H 30/20; A61B 8/54; A61B 5/0095; A61B 8/463; G16H 40/63

(57) ABSTRACT

An information-processing apparatus captures either or both of an ultrasonic image and a photoacoustic image that are imaged by an imaging device, obtains operation information about manipulation of the imaging device for instructing an imaging method and time of the manipulation regarding either or both of the ultrasonic image and the photoacoustic image, obtains time information about either or both of time at which the ultrasonic image is captured and time at which the photoacoustic image is captured, and outputs, to an external device, the operation information, the time information, and either or both of the ultrasonic image and the photoacoustic image that are associated with each other.

### CROSS-REFERENCE TO RELATED APPLICATIONS

This application is a Continuation of International Patent Application No. PCT/JP2017/041405, filed Nov. 17, 2017, which claims the benefit of Japanese Patent Application No. 2016-228064, filed Nov. 24, 2016 and Japanese Patent Application No. 2017-200400, filed Oct. 16, 2017, both of which are hereby incorporated by reference herein in their entirety.

### TECHNICAL FIELD

The present disclosure relates to an information-processing apparatus, a method for processing information, an information-processing system, and a program.

### BACKGROUND ART

An ultrasonic imaging device or a photoacoustic imaging device are used as an imaging device that images a state of the inside of a test object in a minimally invasive manner. The device can capture a video or a still image of an ultrasonic image and a photoacoustic image. The ultrasonic imaging device enables an imaging method called elastography for imaging elastic properties of tissue to be also used. That is, various imaging methods can be used. PTL 1 discloses that a device that can capture the ultrasonic image and the photoacoustic image generates supplementary information about the start address of data of the photoacoustic image and the start address of data of the ultrasonic image in a single frame.

### CITATION LIST

### Patent Literature

PTL 1: Japanese Patent Laid-Open No. 2014-217652

### SUMMARY OF INVENTION

An information-processing apparatus according to an embodiment of the present invention includes a signal-obtaining unit that obtains either or both of a photoacoustic signal that is related to a photoacoustic wave that is generated by irradiating a test object with light and an ultrasonic signal that is related to a reflected wave of an ultrasonic wave with which the test object is irradiated, an information-obtaining unit that obtains operation information about manipulation for obtaining the photoacoustic signal, and an output unit that outputs an object that includes the operation information to an external device.

The information-processing apparatus according to the embodiment of the present invention enables a device that plays a video to obtain information about manipulation for capturing an image from supplementary information. Consequently, workflow of a user who observes the video can be improved.

Further features of the present invention will become apparent from the following description of exemplary embodiments with reference to the attached drawings.

### BRIEF DESCRIPTION OF DRAWINGS

FIG. 1 illustrates an example of the structure of a system that includes an information-processing apparatus according to an embodiment of the present invention.

FIG. 2 illustrates an example of a hardware configuration of the information-processing apparatus according to the embodiment of the present invention.

FIG. 3 illustrates an example of a functional configuration of the information-processing apparatus according to the embodiment of the present invention.

FIG. 4 is a flowchart illustrating an example of a series of processes that are performed by the information-processing apparatus according to the embodiment of the present invention.

FIG. 5 illustrates an example of information that is obtained by the information-processing apparatus according to the embodiment of the present invention.

FIG. 6 illustrates another example of information that is obtained by the information-processing apparatus according to the embodiment of the present invention.

FIG. 7 illustrates another example of information that is obtained by the information-processing apparatus according to the embodiment of the present invention.

FIG. 8 illustrates another example of information that is obtained by the information-processing apparatus according to the embodiment of the present invention.

FIG. 9 illustrates an example of objects that are outputted to external devices by the information-processing apparatus according to the embodiment of the present invention.

FIG. 10 is a timing chart illustrating examples of processes that are performed by the information-processing apparatus according to the embodiment of the present invention.

FIG. 11 is a flowchart illustrating an example of a series of processes that are performed by the information-processing apparatus according to the embodiment of the present invention.

FIG. 12 illustrates an example of information that is obtained by the information-processing apparatus of the embodiment of the present invention.

FIG. 13 is a flowchart illustrating an example of a series of processes that are performed by the information-processing apparatus according to the embodiment of the present invention.

FIG. 14 illustrates an example of an image that is displayed on the basis of information that is obtained by the information-processing apparatus according to the embodiment of the present invention.

### DESCRIPTION OF EMBODIMENTS

Embodiments of the present invention will hereinafter be described with reference to the drawings.

### First Embodiment

In the present disclosure, an acoustic wave that is generated by expansion inside a test object when the test object is irradiated with light is referred to as a photoacoustic wave. An acoustic wave that is emitted form a transducer or a reflected wave (echo) when the emitted acoustic wave is reflected inside the test object is referred to as an ultrasonic wave.

A state of the inside of the test object is imaged in a minimally invasive manner by using an imaging method with ultrasonic waves or an imaging method with photoacoustic waves. In the imaging method with an ultrasonic wave, for example, ultrasonic waves that are emitted from a transducer are reflected by tissue inside the test object depending on a difference between acoustic impedances, and an image is created on the basis of time until reflected waves reach the transducer or the strength of the reflected waves. In the following description, the image that is imaged by using the ultrasonic waves is referred to as an ultrasonic image. A user changes, for example, the angle of a probe during operation and can observe ultrasonic images of various sections in real time. Each ultrasonic image represents the shape of an internal organ or tissue and is used, for example, to find a tumor. In the imaging method with photoacoustic waves, for example, an image is created on the basis of the ultrasonic waves (photoacoustic waves) that are generated by adiabatic expansion of tissue inside the test object that is irradiated with light. In the following description, the image that is imaged by using the photoacoustic waves is referred to as a photoacoustic image. The photoacoustic image represents information that is related to optical properties such as the degree of absorption of light by tissue. For example, it is known that the photoacoustic image can represent a blood vessel by using the optical properties of hemoglobin, and the use of the photoacoustic image is considered, for example, to evaluate the malignancy of a tumor.

In some cases, various kinds of information is collected by imaging different phenomena of the same portion of the test object on the basis of different principles to increase the accuracy of diagnosis. Imaging of the ultrasonic image and imaging of the photoacoustic image are considered, and an imaging device that obtains an image that represents combined characteristics is considered. In particular, the ultrasonic image and the photoacoustic image are imaged by using the ultrasonic waves from the test object, and accordingly, the ultrasonic image and the photoacoustic image can be imaged by the same imaging device. More specifically, the reflected waves and the photoacoustic waves from the irradiated test object can be received by the same transducer. Consequently, an ultrasonic signal and a photoacoustic signal can be obtained by a single probe, and the imaging device that images the ultrasonic image and the photoacoustic image can be achieved without a complex hardware configuration.

In the case where a video is imaged by the imaging device that can capture the ultrasonic image and the photoacoustic image, for example, the photoacoustic image is captured at a position that a user desires while a display unit displays the video of the ultrasonic image. In the case where a doctor carries out diagnosis by referring an image that is captured in a desired imaging method among images that are captured in the above various imaging methods, and the address of data of the ultrasonic image or the photoacoustic image in a single frame is merely added to supplementary information, there is a possibility that the image that is captured in the imaging method that the doctor desires cannot be quickly displayed. That is, it is necessary for the supplementary information to include information that enables the image that is captured by the imaging method that the doctor desires to be quickly displayed. An object of a first embodiment is to quickly identify a section that includes a photoacoustic image when a video is played in the case where the section of the video that includes a series of ultrasonic images includes the photoacoustic image.

Structure of Information-Processing Apparatus

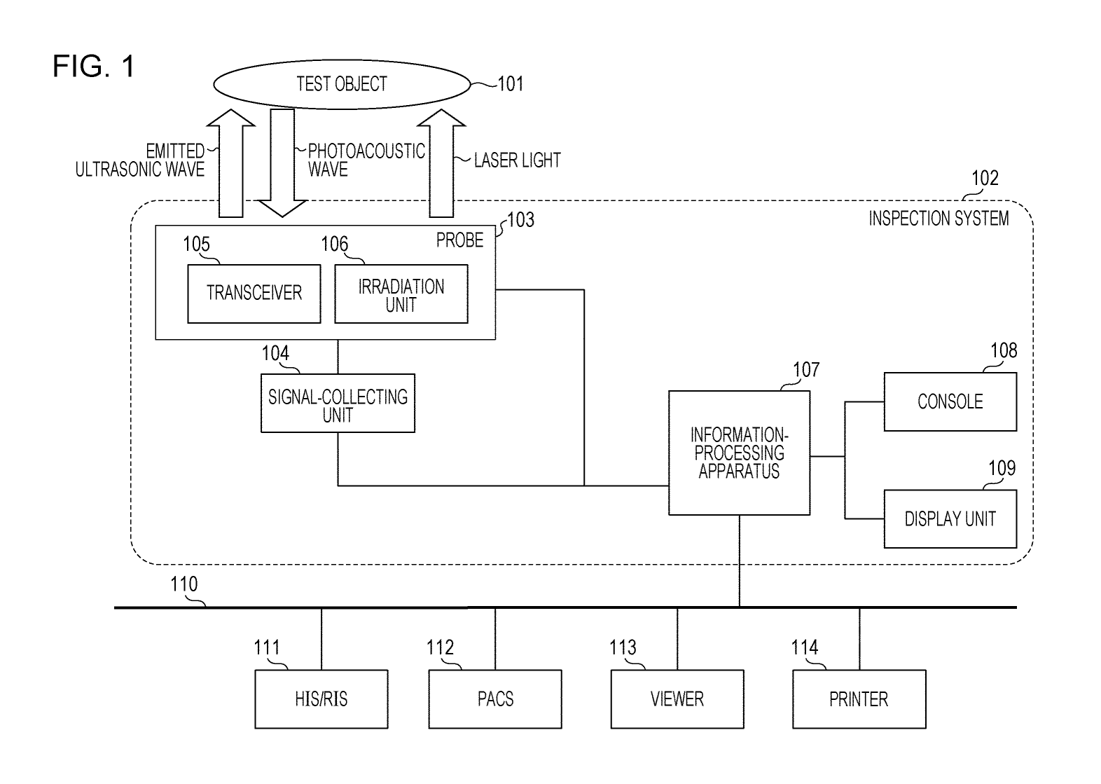

FIG. 1 illustrates an example of the structure of an inspection system 102 that includes an information-processing apparatus 107 according to the first embodiment. The inspection system 102 that can generate the ultrasonic image and the photoacoustic image is connected to various external devices via a network 110. Components that are included in the inspection system 102 and the various external devices do not need to be installed in the same facility provided that the components and the external devices are connected thereto so as to be able to communicate.

The inspection system 102 includes the information-processing apparatus 107, a probe 103, a signal-collecting unit 104, a display unit 109, and a console 108. The information-processing apparatus 107 obtains information about inspection including imaging of the ultrasonic image and photoacoustic image from HIS/RIS 111 and controls the probe 103 and the display unit 109 during the inspection. The information-processing apparatus 107 obtains the ultrasonic signal and the photoacoustic signal from the probe 103 and the signal-collecting unit 104. The information-processing apparatus 107 captures the ultrasonic image on the basis of the ultrasonic signal and captures the photoacoustic image on the basis of the photoacoustic signal. The information-processing apparatus 107 may capture a superimposed image that is obtained by superimposing the photoacoustic image on the ultrasonic image. The information-processing apparatus 107 transmits information to and receives information from the external devices such as the HIS/RIS 111 and a PACS 112 in accordance with standards such as HL7 (Health level 7) and DICOM (Digital Imaging and Communications in Medicine).

Examples of an inner region of a test object 101 the ultrasonic image of which is imaged by the inspection system 102 include a circulatory organ region, the breast, the liver, the pancreas, and the abdomen. For example, the inspection system 102 may image the ultrasonic image of the test object to which an ultrasonic contrast agent with micro bubbles is given.

Examples of the inner region of the test object the photoacoustic image of which is imaged by the inspection system 102 include a circulatory organ region, the breast, the groin, the abdomen, and the limbs that include the fingers and the toes. In particular, the target of the photoacoustic image to be imaged may include a blood vessel region that includes a new blood vessel and plaque on a blood vessel wall depending on characteristics that are related to light absorption inside the test object. The inspection system 102 may image the photoacoustic image of the test object 101 to which a contrast agent of a pigment such as methylene blue or indocyanine green, gold granules, an accumulation thereof, or a substance that is chemically modified is given.

The probe 103 is operated by the user and transmits the ultrasonic signal and the photoacoustic signal to the signal-collecting unit 104 and the information-processing apparatus 107. The probe 103 is controlled by an imaging control unit 302. The user can control the probe 103 by using an input unit (not illustrated) that is included in the probe 103 such as a freeze button. The probe 103 transmits information about a manipulation input of the user to the information-processing apparatus 107. The probe 103 includes a transceiver 105 and an irradiation unit 106. The probe 103 emits the ultrasonic waves from the transceiver 105 and receives the reflected waves by the transceiver 105. The probe 103 irradiates the test object with light from the irradiation unit 106 and receives the photoacoustic waves by the transceiver 105. The probe 103 is preferably controlled such that the ultrasonic waves are emitted to obtain the ultrasonic signal and the light is emitted to obtain the photoacoustic signal when information that represents contact with the test object is received.

The transceiver 105 includes at least one transducer (not illustrated), a matching layer (not illustrated), a damper (not illustrated), and an acoustic lens (not illustrated). The transducer (not illustrated) is composed of a substance that has a piezoelectric effect such as PZT (lead zirconate titanate) or PVDF (polyvinylidene difluoride). The transducer (not illustrated) may not be a piezoelectric element, and examples thereof include a capacitive micro-machined ultrasonic transducer (CMUT) and a transducer with a Fabry-Perot interferometer. The ultrasonic signal typically has a frequency component at 2 to 20 MHz. The photoacoustic signal has a frequency component at 0.1 to 100 MHz. For example, the transducer (not illustrated) can detect the frequency. A signal that is obtained by the transducer (not illustrated) is a time-resolved signal. The amplitude of the signal that is received represents a value based on sound pressure that is applied to the transducer at a time. The transceiver 105 includes a control unit or a circuit (not illustrated) for electronic focus. The transducer (not illustrated) is formed into a sector, a linear array, a convex shape, an annular array, or a matrix array. The probe 103 obtains the ultrasonic signal and the photoacoustic signal. The probe 103 may alternately obtain the ultrasonic signal and the photoacoustic signal, may obtain the ultrasonic signal and the photoacoustic signal at the same time, or may obtain the ultrasonic signal and the photoacoustic signal in a predetermined manner.

The transceiver 105 may include an amplifier (not illustrated) that amplifies time-series analog signals that are received by the transducer (not illustrated). The transducer (not illustrated) may be divided into a transmitter and a receiver in accordance with the purpose of imaging of the ultrasonic image. Alternatively, the transducer (not illustrated) may be divided into an ultrasonic-image transducer and a photoacoustic-image transducer.

The irradiation unit 106 includes a light source (not illustrated) for obtaining the photoacoustic signal and an optical system (not illustrated) that guides pulsed light that is emitted from the light source (not illustrated) to the test object. The pulse width of the light that is emitted from the light source (not illustrated) is, for example, no less than 1 ns and no more than 100 ns. The wavelength of the light that is emitted from the light source (not illustrated) is, for example, no less than 400 nm and no more than 1600 nm. In the case where a blood vessel near a surface of the test object is imaged with high resolution, the wavelength is preferably no less than 400 nm and no more than 700 nm at which the light is greatly absorbed in the blood vessel. In the case where a deep portion of the test object is imaged, the wavelength is preferably no less than 700 nm and no more than 1100 nm at which the light is unlikely to be absorbed by water and tissue such as fat.

Examples of the light source (not illustrated) include a laser and a light-emitting diode. The irradiation unit 106 may be a light source that can change the wavelength in order to obtain the photoacoustic signal by using light at wavelengths. Alternatively, the irradiation unit 106 may include light sources that emit light at different wavelengths, where the light at the different wavelengths can be emitted from the light sources. Examples of the laser include a solid-state laser, a gas laser, a pigment laser, and a semiconductor laser. The light source (not illustrated) may be a pulse laser such as a Nd:YAG laser or an alexandrite laser. Alternatively, the light source (not illustrated) may be an OPO (optical parametric oscillator) laser or a Ti:sa laser that changes the light of the Nd:YAG laser into excitation light. Alternatively, the light source (not illustrated) may be a microwave source.

Optical elements such as a lens, a mirror, and an optical fiber are used as the optical system (not illustrated). In the case where the test object is the breast, the beam diameter of the pulsed light that is emitted is preferably increased. Accordingly, the optical system (not illustrated) may include a diffuser panel that diffuses the emitted light. Alternatively, the optical system (not illustrated) may include, for example, the lens and may be capable of focusing a beam in order to increase the resolution.

The signal-collecting unit 104 convers the analog signals of the photoacoustic waves and the reflected waves that are received by the probe 103 into digital signals. The signal-collecting unit 104 transmits the ultrasonic signal and the photoacoustic signal that are converted into the digital signals to the information-processing apparatus 107.

The display unit 109 displays information about the image that is imaged by the inspection system 102 and the inspection in response to control of the information-processing apparatus 107. The display unit 109 provides an interface for receiving a user instruction in response to control of the information-processing apparatus 107. An example of the display unit 109 is a liquid-crystal display.

The console 108 transmits information about the manipulation input of the user to the information-processing apparatus 107. Examples of the console 108 include a keyboard, a track ball, or various buttons for the manipulation input that is related to the inspection.

The display unit 109 and the console 108 may be integrated into a touch panel display. The information-processing apparatus 107, the display unit 109, and the console 108 do not need to be different devices but may be integrated into an operator console. The information-processing apparatus 107 may include plural probes.

The HIS/RIS 111 manages information about patients and information about the inspection. The HIS (Hospital Information System) assists services of a hospital. The HIS includes an electronic medical record system, an ordering system, and a medical accounting system. The RIS (Radiology Information System) manages inspection information in a radiology department to manage the progress of the inspection by the imaging device. The inspection information includes an inspection ID for identification and information about an imaging technique that is included in the inspection. Ordering systems that are built in respective departments may be connected to the inspection system 102 instead of the RIS or in addition to the RIS. The inspection is collectively managed from an order to payment by the HIS/RIS 111. The HIS/RIS 111 transmits information about the inspection that is carried out by the inspection system 102 to the information-processing apparatus 107 in response to an inquiry from the information-processing apparatus 107. The HIS/RIS 111 receives information about the progress of the inspection from the information-processing apparatus 107. The HIS/RIS 111 performs a process for the payment when the HIS/RIS 111 receives information that the inspection is finished from the information-processing apparatus 107.

The PACS (Picture Archiving and Communication System) 112 is a database system that holds images that are captured by various imaging devices inside and outside the facility. The PACS 112 includes a storage unit (not illustrated) that stores medical images and supplementary information about imaging conditions of the medical images, parameters of an imaging process that includes reconfiguration, and the patients, and a controller (not illustrated) that manages the information that is stored in the storage unit. The PACS 112 stores the ultrasonic image, the photoacoustic image, and the superimposed image, which are objects that are outputted from the information-processing apparatus 107. The communication between the PACS 112 and the information-processing apparatus 107 and the various images that are stored in the PACS 112 preferably satisfy the standards such as the HL7 and the DICOM. The various images that are outputted from the information-processing apparatus 107 are stored with the supplementary information associated with various tags in accordance with the DICOM standard.

A viewer 113 is a terminal for image diagnosis, reads the images that are stored in, for example, the PACS 112, and displays the images for the diagnosis. A doctor observes the images that are displayed on the viewer 113 and records an image diagnosis report of information that is obtained by the observation. The image diagnosis report that is created by using the viewer 113 may be stored in the viewer 113 or may be outputted to the PACS 112 or a report server (not illustrated) and stored.

A printer 114 prints the images that are stored in, for example, the PACS 112. An example of the printer 114 is a film printer, which outputs the images by printing the images that are stored in, for example, the PACS 112 on a film.

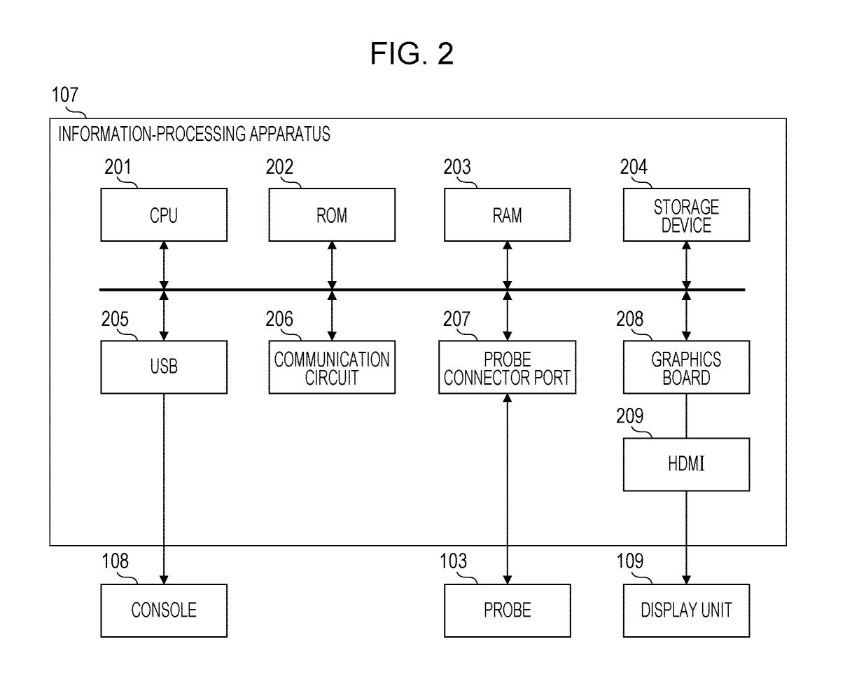

FIG. 2 illustrates an example of a hardware configuration of the information-processing apparatus 107. An example of the information-processing apparatus 107 is a computer. The information-processing apparatus 107 includes a CPU 201, a ROM 202, a RAM 203, a storage device 204, a USB 205, a communication circuit 206, a probe connector port 207, and a graphics board 208. These are connected so as to be able to communicate by using a BUS. The BUS is used to transmit and receive data between pieces of hardware that are connected to each other and to transmit instructions from the CPU 201 to another hardware.

The CPU (Central Processing Unit) 201 is a control circuit that comprehensively controls the information-processing apparatus 107 and components that are connected thereto. The CPU 201 executes programs that are stored in the ROM 202 for the control. The CPU 201 executes a display driver, which is software for controlling the display unit 109, for display control of the display unit 109. The CPU 201 controls input and output for the console 108.

The ROM (Read Only Memory) 202 stores a program in which control procedures of the CPU 201 are written, and data. The ROM 202 stores a boot program of the information-processing apparatus 107 and various initial data. In addition, various programs for the processes of the information-processing apparatus 107 are stored therein.

The RAM (Random Access Memory) 203 provides a working memory area when the CPU 201 executes an instruction program for the control. The RAM 203 has stack and a working area. The RAM 203 stores programs for performing the processes of the information-processing apparatus 107 and the components that are connected thereto, and various parameters that are used for the imaging process. The RAM 203 stores a control program that is executed by the CPU 201 and temporally stores various kinds of data for various kinds of control of the CPU 201.

The storage device 204 is an auxiliary storage device that saves various kinds of data such as the ultrasonic image and the photoacoustic image. Examples of the storage device 204 include a HDD (Hard Disk Drive) and a SSD (Solid State Drive).

The USB (Universal Serial Bus) 205 is a connector that is connected to the console 108.

The communication circuit 206 is a circuit for communication with various external devices that are connected to the components of the inspection system 102 and the network 110. For example, the communication circuit 206 outputs information that is contained in a transfer packet to the external devices via the network 110 by using a communication technique such as TCP/IP. The information-processing apparatus 107 may include plural communication circuits to fit a desired communication form.

The probe connector port 207 connects the probe 103 to the information-processing apparatus 107.

The graphics board 208 includes a GPU (Graphics Processing Unit) and a video memory. For example, the GPU makes calculations that are related to a reconfiguration process for generating the photoacoustic image from the photoacoustic signal.

A HDMI (registered trademark) (High Definition Multimedia Interface) 209 is a connector that is connected to the display unit 109.

The CPU 201 and the GPU are examples of a processor. The ROM 202, the RAM 203, and the storage device 204 are examples of a memory. The information-processing apparatus 107 may include plural processors. According to the first embodiment, the processor of the information-processing apparatus 107 executes the programs that are stored in the memory to perform the functions of the components of the information-processing apparatus 107.

The information-processing apparatus 107 may include a CPU, a GPU, and an ASIC (Application Specific Integrated Circuit) that exclusively perform a specific process. The information-processing apparatus 107 may include a FPGA (Field-Programmable Gate Array) in which the specific process or all of the processes are programed.

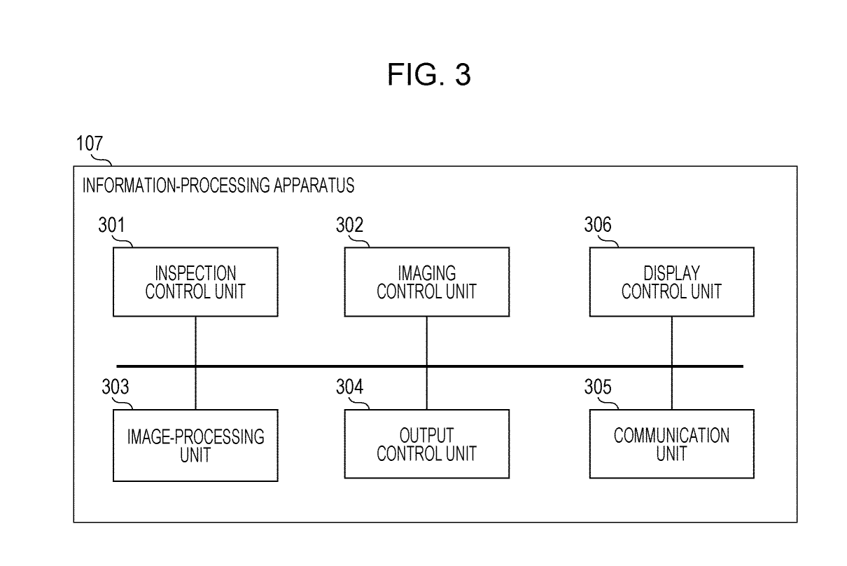

FIG. 3 illustrates an example of a functional configuration of the information-processing apparatus 107. The information-processing apparatus 107 includes an inspection control unit 301, the imaging control unit 302, an image-processing unit 303, an output control unit 304, a communication unit 305, and a display control unit 306.

The inspection control unit 301 obtains information about the order for the inspection from the HIS/RIS 111. The order for the inspection includes information about the patient to be inspected and information about the imaging technique. The inspection control unit 301 transmits the information about the order for the inspection to the imaging control unit 302. The inspection control unit 301 causes the display unit 109 to display the information about the inspection to provide the user with the information about the inspection via the display control unit 306. The information about the inspection that is displayed on the display unit 109 includes information about the patient to be inspected, the information about the imaging technique that is included in the inspection, and the image that has been imaged and generated. The inspection control unit 301 transmits the information about the progress of the inspection to the HIS/RIS 111 via the communication unit 305.

The imaging control unit 302 controls the probe 103 on the basis of the information about the imaging technique that is received from the inspection control unit 301 and obtains the ultrasonic signal and the photoacoustic signal from the probe 103 and the signal-collecting unit 104. The imaging control unit 302 instructs the irradiation unit 106 to emit light. The imaging control unit 302 instructs the transceiver 105 to emit the ultrasonic waves. The imaging control unit 302 instructs the irradiation unit 106 and the transceiver 105 on the basis of the information about the manipulation input of the user and the imaging technique. The imaging control unit 302 instructs the transceiver 105 to receive the ultrasonic waves. The imaging control unit 302 instructs the signal-collecting unit 104 to sample the signals. The imaging control unit 302 controls the probe 103 as described above and obtains the ultrasonic signal and the photoacoustic signal separately. The imaging control unit 302 is an example of the information-obtaining unit that obtains timing information. The imaging control unit 302 also obtains operation information about the manipulation input of the user during the inspection. The user can provide the manipulation input that is related to imaging of the ultrasonic image and the photoacoustic image by using a user interface that is displayed on the display unit 109. The imaging control unit 302 obtains the operation information of the user for the information-processing apparatus 107. The operation information of the user for the probe 103 is also obtained from the probe 103. That is, the imaging control unit 302 is an example of the information-obtaining unit that obtains the operation information.

The imaging control unit 302 may also obtain information (referred to below timing information) about timing with which the ultrasonic signal and the photoacoustic signal are obtained. The timing information represents, for example, timing with which the imaging control unit 302 controls the probe 103 to emit light and the ultrasonic waves. The information that represents the timing may be time or elapsed time after the inspection is started. The imaging control unit 302 obtains the ultrasonic signal and the photoacoustic signal that are converted into digital signals and that are outputted from the signal-collecting unit 104. That is, the imaging control unit 302 is an example of a signal-obtaining unit that obtains the ultrasonic signal and the photoacoustic signal. The imaging control unit 302 is an example of the information-obtaining unit that obtains the timing information.

The image-processing unit 303 generates the ultrasonic image, the photoacoustic image, and the superimposed image that is obtained by superimposing the photoacoustic image on the ultrasonic image. The image-processing unit 303 generates a video that includes the ultrasonic image and the photoacoustic image.

Specifically, the image-processing unit 303 generates the photoacoustic image on the basis of the photoacoustic signal that is obtained by the imaging control unit 302. The image-processing unit 303 reconfigures distribution (referred to below as initial sound pressure distribution) of acoustic waves when light is emitted on the basis of the photoacoustic signal. The image-processing unit 303 obtains absorption coefficient distribution of light inside the test object by dividing the reconfigured initial sound pressure distribution by light fluence distribution of the test object with respect to the light with which the test object is irradiated. The fact that the degree of absorption of light inside the test object varies depending on the wavelength of the light with which the test object is irradiated is applied to obtain concentration distribution of a substance inside the test object from the absorption coefficient distribution relative to wavelengths. For example, the image-processing unit 303 obtains the concentration distribution of oxyhemoglobin and deoxyhemoglobin in the substance inside the test object. The image-processing unit 303 also obtains oxygen saturation distribution as a ratio of oxyhemoglobin concentration to deoxyhemoglobin concentration. For example, the photoacoustic image that is generated by the image-processing unit 303 represents information about any one of or all of the initial sound pressure distribution, the light fluence distribution, the absorption coefficient distribution, the concentration distribution of the substance, and the oxygen saturation distribution, described above.

The image-processing unit 303 obtains a spectral line that is obtained by converting the amplitude of the reflected wave of the ultrasonic signal into luminance and generates the ultrasonic image (B-mode image) by changing the position at which the spectral line is displayed so as to fit scanning of an ultrasonic beam. In the case where the probe 103 is a three-dimensional probe, the image-processing unit 303 can generate the ultrasonic image (C-mode image) that includes three sections that are perpendicular to each other. The image-processing unit 303 generates the image of a freely selected section and a three-dimensional image after rendering on the basis of a three-dimensional ultrasonic image. The image-processing unit 303 is an example of the image-capturing unit that captures either or both of the ultrasonic image and the photoacoustic image.

The output control unit 304 generates objects for transmitting the various kinds of information to the external devices such as the PACS 112 and the viewer 113 in response to the control of the inspection control unit 301 and the manipulation input of the user. The objects correspond to information to be transmitted to the external devices such as the PACS 112 and the viewer 113 from the information-processing apparatus 107. For example, the output control unit 304 generates DICOM objects for outputting, to the PACS 112, the ultrasonic image, the photoacoustic image, and the superimposed image thereof that are generated by the image-processing unit 303. The objects that are outputted to the external devices include the supplementary information as the various tags in accordance with the DICOM standard. For example, the supplementary information includes the patient information, information that represents the imaging device that images the above images, an image ID for identification of the images, the inspection ID for identification of the inspection during which the above images are imaged, and information about the probe 103.

The supplementary information that is generated by the output control unit 304 includes operation information about the manipulation input of the user during the inspection.

The communication unit 305 controls transmission and reception of information between the information-processing apparatus 107 and the external devices such as the HIS/RIS 111, the PACS 112, and the viewer 113 via the network 110. A transmitting and receiving control unit receives the information about the order for the inspection from the HIS/RIS 111. The transmitting and receiving control unit transmits the objects that are generated by an imaging-failure-process control unit to the PACS 112 and the viewer 113.

The display control unit 306 controls the display unit 109 to cause the display unit 109 to display the information. The display control unit 306 causes the display unit 109 to display the information in response to input from another module or the manipulation input of the user by using the console 108. The display control unit 306 is an example of the display-controlling unit.

Series of Processes of Information-Processing Apparatus 107

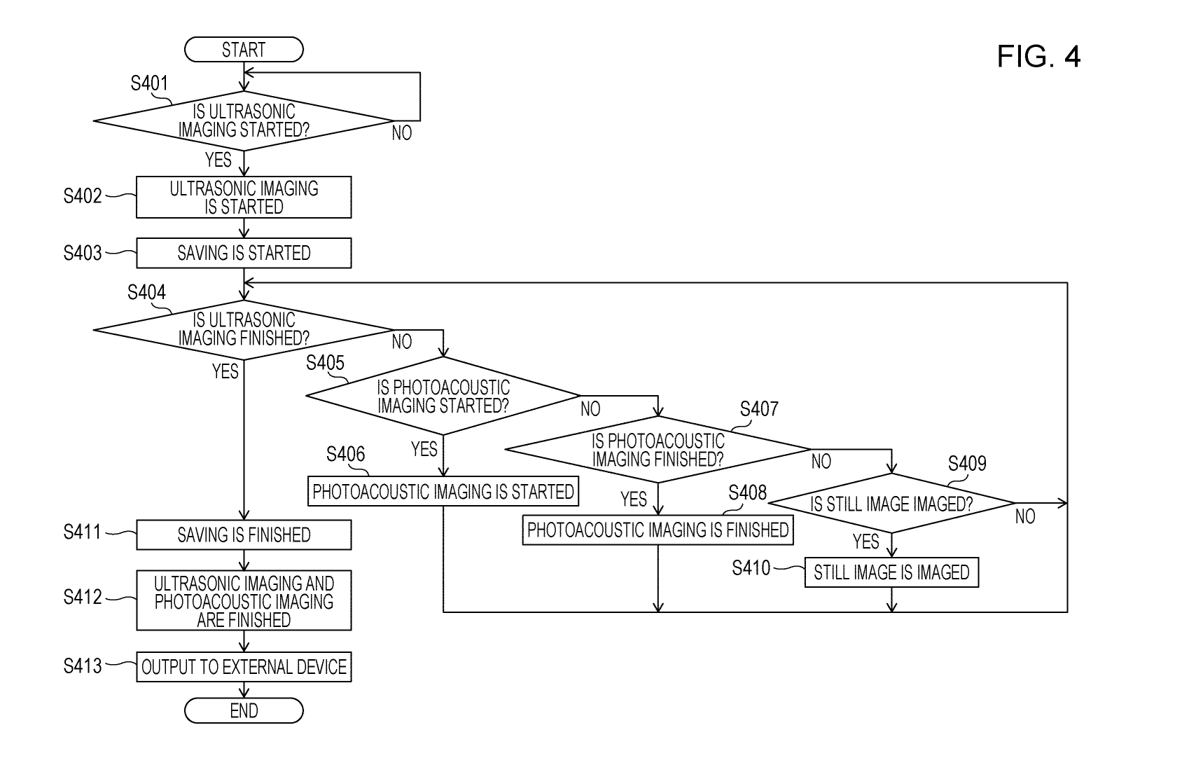

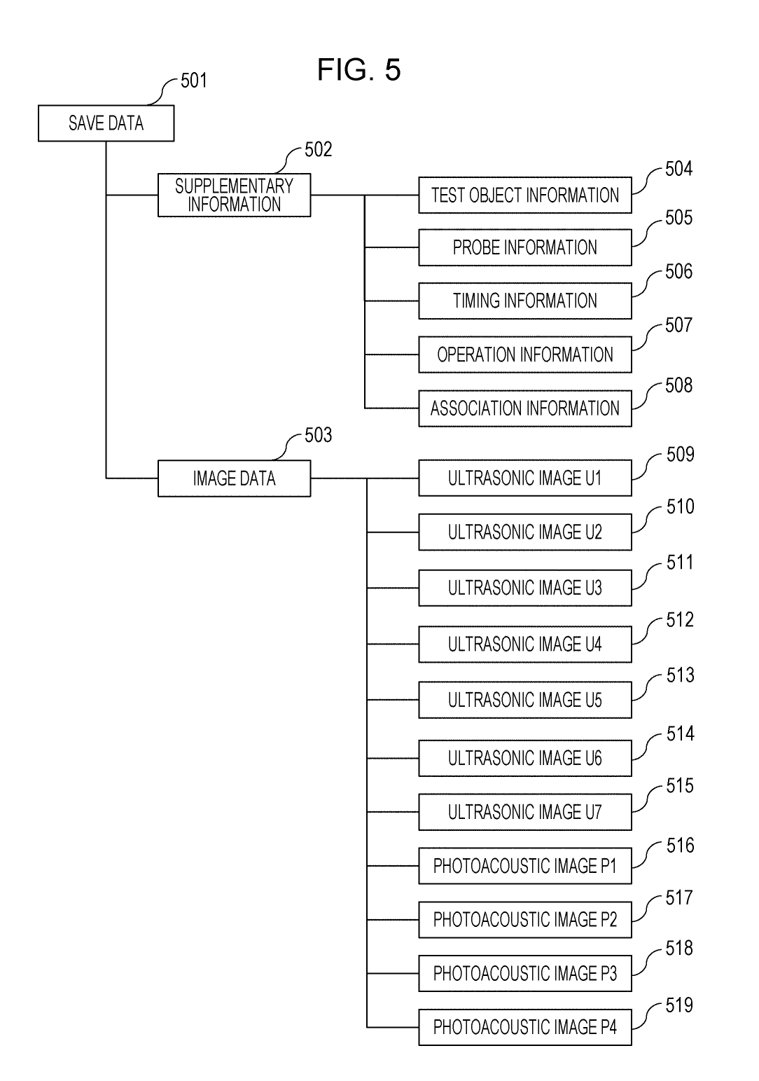

FIG. 4 is a flowchart illustrating an example of a series of processes of the information-processing apparatus 107 to image the video that includes the ultrasonic image and the photoacoustic image, generate the supplementary information, and output the objects that include the video and the supplementary information to the external devices. In an example described below, the photoacoustic image is imaged on the basis of the manipulation input of the user while the ultrasonic image is imaged. The processes described below are performed mainly by the CPU 201 or the GPU unless otherwise particularly described. The information that is obtained by the information-processing apparatus 107 will be described with reference to FIG. 5 to FIG. 9 appropriately.

At a step S401, the inspection control unit 301 receives an instruction to start imaging. The inspection control unit 301 first obtains the information about the order for the inspection from the HIS/RIS 111. The display control unit 306 causes the display unit 109 to display the information about the inspection that is represented by the order for the inspection and the user interface into which the user inputs an instruction for the inspection. Imaging is started in response to the instruction that is inputted into the user interface by using the console 108 for start of imaging. Imaging of the ultrasonic image is started on the basis of the manipulation input of the user or automatically.

At a step S402, the imaging control unit 302 controls the probe 103 and the signal-collecting unit 104 to start imaging of the ultrasonic image. The user presses the probe 103 against the test object 101 for imaging at a desired position. The imaging control unit 302 obtains the ultrasonic signal, which is a digital signal, and the timing information about obtaining of the ultrasonic signal and stores these in the RAM 203. The image-processing unit 303 generates the ultrasonic image by performing a process such as delay and sum on the ultrasonic signal. The ultrasonic signal that is saved in the RAM 203 may be deleted after the ultrasonic image is generated. The image-processing unit 303 causes the display unit 109 to display the captured ultrasonic image by using the display control unit 306. The imaging control unit 302 and the image-processing unit 303 repeat these steps to update the ultrasonic image that is to be displayed on the display unit 109. Consequently, the video that includes each updated ultrasonic image is displayed.

At a step S403, the output control unit 304 starts a process of saving image data that is obtained by the image-processing unit 303 and the supplementary information. Start of saving is instructed by the manipulation input into the information-processing apparatus 107 or the probe 103.

At a step S404, the imaging control unit 302 receives an instruction to finish ultrasonic imaging. During the inspection, the display control unit 306 causes the display unit 109 to display the user interface for the manipulation input that is related to the inspection. The user can instruct to finish the ultrasonic imaging by the manipulation input into the user interface. In another example, the user can instruct to finish the ultrasonic imaging by the manipulation input into an input unit (not illustrated) of the probe 103. When the instruction for the finish is received, the flow proceeds to a step S411. In the case where there is no instruction, the flow proceeds to a step S405.

At the step S405, the imaging control unit 302 receives an instruction to start photoacoustic imaging. The user can instruct to start the photoacoustic imaging by the manipulation input that is related to the inspection into the user interface or the manipulation input into the probe 103. When the instruction for the start is received, the flow proceeds to a step S406. In the case where there is no instruction, the flow proceeds to a step S407.

At the step S404 and the step S405, the imaging control unit 302 obtains the operation information that represents manipulation of the imaging device to instruct the imaging method and the time of the manipulation. From this perspective, the imaging control unit 302 is an example of the information-obtaining unit.

At the step S406, the imaging control unit 302 controls the probe 103 and the signal-collecting unit 104 to start imaging of the photoacoustic image. The user presses the probe 103 against the test object 101 for imaging at a desired position. The imaging control unit 302 obtains the photoacoustic signal, which is a digital signal, and the timing information about obtaining of the photoacoustic signal and stores these in the RAM 203. The image-processing unit 303 generates the photoacoustic image by performing a process such as universal back-projection (UBP) on the photoacoustic signal. The photoacoustic signal that is saved in the RAM 203 may be deleted after the photoacoustic image is generated. The image-processing unit 303 causes the display unit 109 to display the captured photoacoustic image by using the display control unit 306. The imaging control unit 302 and the image-processing unit 303 repeat these steps to update the photoacoustic image that is to be displayed on the display unit 109. Consequently, the video that includes each updated photoacoustic image is displayed. In the case where the flow proceeds from the step S406 to the step S404 and the imaging control unit 302 receives the instruction to finish the ultrasonic imaging at the step S404, the imaging control unit 302 controls the probe 103 to finish the photoacoustic imaging.

At a step S407, the imaging control unit 302 receives an instruction to finish the photoacoustic imaging. The user can instruct to finish the photoacoustic imaging by the manipulation input that is related to the inspection into the user interface or the manipulation input into the probe 103. When the instruction for the finish is received, the flow proceeds to a step S408. In the case where there is no instruction, the flow proceeds to a step S409.

At the step S405 and the step S407, since the manipulation input that is related to imaging of the photoacoustic image is provided by the user, the imaging control unit 302 obtains the operation information.

At the step S408, the imaging control unit 302 controls the probe 103 to finish imaging of the photoacoustic image.

At the step S409, the imaging control unit 302 receives an instruction to image a still image. The user can instruct to image the still image by the manipulation input that is related to the inspection into the user interface or the manipulation input into the probe 103. The still image may be a still image of the ultrasonic image, may be a still image of the photoacoustic image, or may be a still image of the superimposed image that is obtained by superimposing the photoacoustic image on the ultrasonic image. When the instruction to image the still image is received, the flow proceeds to a step S410. When there is no instruction, the flow proceeds to the step S404.

At the step S410, the imaging control unit 302 controls the probe 103 and the signal-collecting unit 104 to perform a process of imaging the still image. The imaging control unit 302 controls the probe 103 and the signal-collecting unit 104 in conditions such as an operation mode proper to imaging of the still image and a sampling period. The processes of capturing the ultrasonic image and the photoacoustic image by the image-processing unit 303 are the same as the processes described for the step S402 and the step S408.

In the processes from the step S404 to the step S410, the imaging control unit 302 obtains timing information on the ultrasonic image and the photoacoustic image. The timing information on the ultrasonic image is related to timing with which the ultrasonic signal that is used for the ultrasonic image is obtained. In the case where the ultrasonic signals are used for the ultrasonic image, the timing information may be related to timing with which any one of the ultrasonic signals is obtained provided that management is united with each ultrasonic image that is captured during the inspection. The timing with which the ultrasonic signal is obtained may be timing with which the information-processing apparatus 107 receives the ultrasonic signal, may be timing with which the ultrasonic waves are emitted from the probe 103 to the test object 101, may be timing with which the probe 103 receives the ultrasonic waves, may be timing with which a driving signal to the probe 103 for emission and reception of the ultrasonic waves is detected, or may be timing with which the signal-collecting unit 104 receives the ultrasonic signal. The timing information on the photoacoustic image is related to timing with which the photoacoustic signal that is used for the photoacoustic image is obtained. In the case where the photoacoustic signals are used for the photoacoustic image, the timing information may be related to timing with which any one of the photoacoustic signals is obtained provided that management is united with each photoacoustic image that is captured during the inspection. The timing with which the photoacoustic signal is obtained may be timing with which the information-processing apparatus 107 receives the photoacoustic signal, may be timing with which the probe 103 irradiates the test object 101 with light, may be timing with which the probe 103 receives the photoacoustic waves, may be timing with which a driving signal to the probe 103 for emission of light or reception of the photoacoustic waves is detected, or may be timing with which the signal-collecting unit 104 receives the photoacoustic signal.

That is, the imaging control unit 302 obtains the timing information (time information) about either or both of the time at which the ultrasonic image is captured and the time at which the photoacoustic image is captured. From this perspective, the imaging control unit 302 is an example of the information-obtaining unit.

At the step S411, the output control unit 304 saves the information that is obtained at the step S403 to the step S411 and finishes a process that is related to saving.

FIG. 5 illustrates an example of the structure of data that is obtained in the process that is related to saving, that starts at the S403 and that finishes at the step S411. Save data 501 is saved in the storage device 204. The save data 501 includes supplementary information 502 and image data 503. For example, the supplementary information 502 is recorded in header of the save data 501.

The image data 503 includes ultrasonic images 509 to 515 and photoacoustic images 516 to 519 that are captured at the step S403 to the step S411. In an example illustrated in FIG. 5, the ultrasonic images 509 to 515 have respective identifiers U1 to U7 for identification. The photoacoustic images 516 to 519 have respective identifiers P1 to P4 for identification.

The supplementary information 502 includes test object information 504 that represents the attribute of the test object 101, probe information 505 about the probe 103 that is used for imaging, timing information 506, operation information 507, and association information 508.

The test object information 504 includes, for example, information about any one of or all of a test object ID, a test object name, an age, blood pressure, a heart rate, a body temperature, a height, a weight, anamnesis, the week of pregnancy, and the inspection. In the case where the inspection system 102 includes an electrocardiograph (not illustrated) and a pulse oximeter (not illustrated), information about electrocardiogram and oxygen saturation may be saved as the test object information 504.

The probe information 505 includes the information about the probe 103 such as the type of the probe 103 and the position and inclination thereof during imaging. The inspection system 102 may include a magnetic sensor (not illustrated) that detects the position and inclination of the probe 103. The imaging control unit 302 may obtain the information from the magnetic sensor (not illustrated).

The timing information 506 is related to timing with which the ultrasonic images 509 to 515 and the photoacoustic images 516 to 519 are captured.

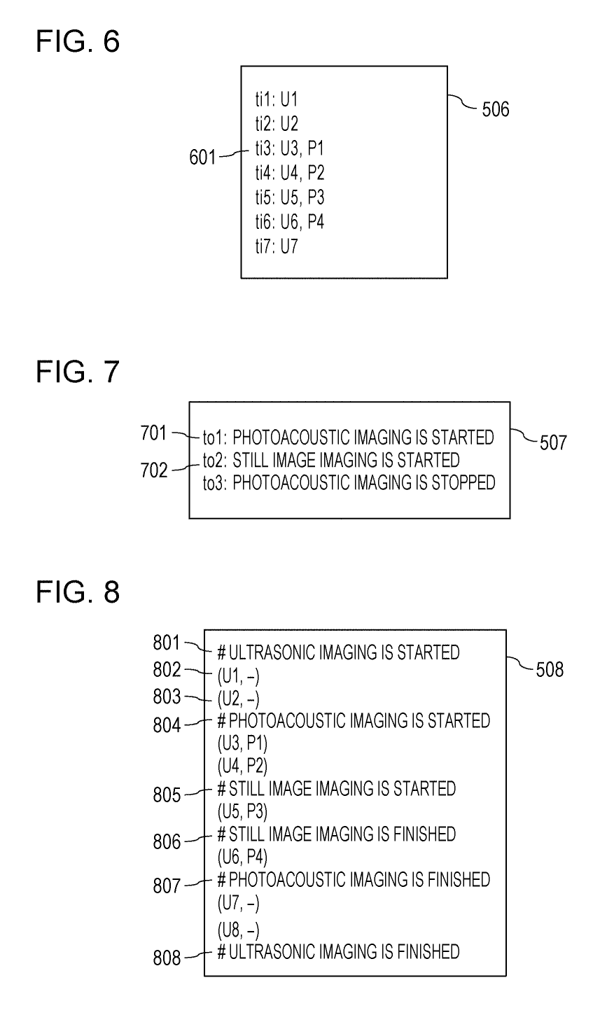

FIG. 6 illustrates an example of the timing information 506. Time and the identifier of an image frame that is obtained at the time are recorded in a time-series order in the rows of the timing information 506. For example, a row 601 represents that a frame U3 of the ultrasonic image and a frame P1 of the photoacoustic image are obtained at time ti3.

The operation information 507 is about the manipulation input of the user when the ultrasonic images 509 to 515 and the photoacoustic images 516 to 519 are captured.

FIG. 7 illustrates an example of the operation information 507. Time and the content of the manipulation that is instructed at the time are recorded in a time-series order in the rows of the operation information 507. For example, a row 701 represents that start of the photoacoustic imaging is instructed at time tot. For example, timing of the manipulation input of the user by using the console 108 is recorded as instruction time.

The association information 508 represents a relationship between timing with which the ultrasonic images 509 to 515 and the photoacoustic images 516 to 519 are captured and timing of the manipulation input of the user.

FIG. 8 illustrates an example of the association information 508. The manipulation input of the user or the identifier of the obtained image is recorded in a time-series order in the rows of the association information 508. (Um, Pn) represents that a frame Um of the ultrasonic image and a frame Pn of the photoacoustic image are substantially simultaneously obtained. (Um, −) represents that only the frame Um of the ultrasonic image is obtained with certain timing. Rows that begin with a mark “#” represent the content of the manipulation input of the user. For example, rows 801 to 804 represent that the frames U1 and U2 of the ultrasonic image are obtained in order right after the instruction to start the ultrasonic imaging and that the instruction to start the photoacoustic imaging is subsequently provided.

The association information 508 can include virtual manipulation input that does not entail the manipulation input of the user. The virtual manipulation input is automatically provided by the apparatus and represents logical phenomena such as the progress and finish of the processes in the case where the information-processing apparatus 107 performs a series of processes with the manipulation input of the user acting as a trigger. For example, “# still image imaging is finished” in a row 806 is the virtual manipulation input and represents finish of the process that is related to the still image imaging and that is performed with the instruction to start the still image imaging in a row 805 acting as a trigger. The virtual manipulation input is automatically inserted into the association information 508 by the output control unit 304.

At a step S412, the imaging control unit 302 controls the probe 103 to finish imaging of the ultrasonic image and imaging of the photoacoustic image.

At a step S413, the output control unit 304 generates an object for output to the external device on the basis of the information that is saved up to the step S411. The communication unit 305 outputs the object to the external device such as the PACS 112.

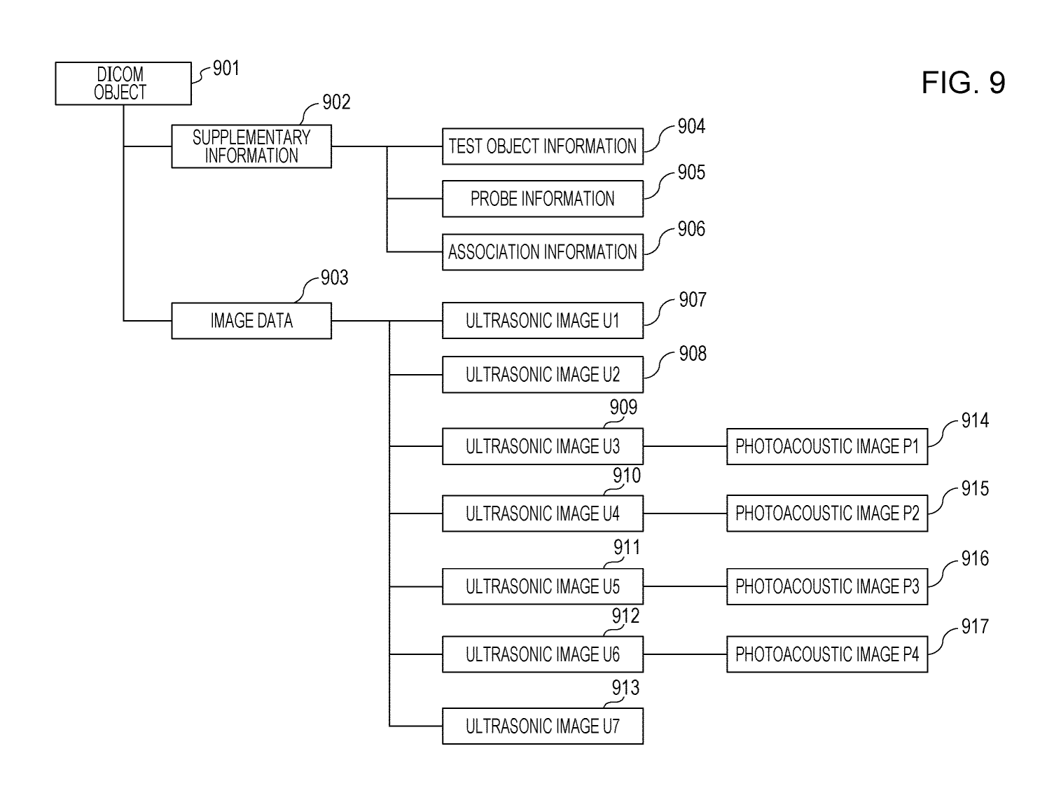

FIG. 9 illustrates an example of the object that is generated at the step S413. A DICOM object 901 includes supplementary information 902 and image data 903. The supplementary information 902 is written, for example, in the header of the image data 903.

The supplementary information 902 includes test object information 904, probe information 905, and association information 906. The test object information 904 corresponds to the test object information 504 illustrated in FIG. 5. The probe information 905 corresponds to the probe information 505 illustrated in FIG. 5. The association information 906 corresponds to the association information 508 illustrated in FIG. 5. The information that is included in the supplementary information 902 may include the same information as the corresponding information illustrated in FIG. 5, may include only essential information for the DICOM standard, or may include only a freely predetermined item. For example, the test object information 904 includes only the test object ID, the age, the gender, and the inspection ID. The supplementary information 902 may not include the probe information 905. The supplementary information 902 may also include timing information that corresponds to the timing information 506 illustrated in FIG. 5 and operation information that corresponds to the operation information 507 but this is not essential because the association information 906 includes the operation information and the timing information.

The image data 903 includes ultrasonic images 907 to 913 and photoacoustic images 914 to 917. In an example illustrated in FIG. 9, the photoacoustic images 914 to 917 are overlay images that are associated with the respective ultrasonic images 909 to 912.

The photoacoustic image may be separated from the DICOM object 901 to use the photoacoustic image as an example of another DICOM object such as a CSPS (Color Softcopy Presentation State). In the case where the CSPS is used, the output control unit 304 may convert the photoacoustic image into an annotation object. In another example, the superimposed image of the ultrasonic image and the photoacoustic image may be a secondary capture image.

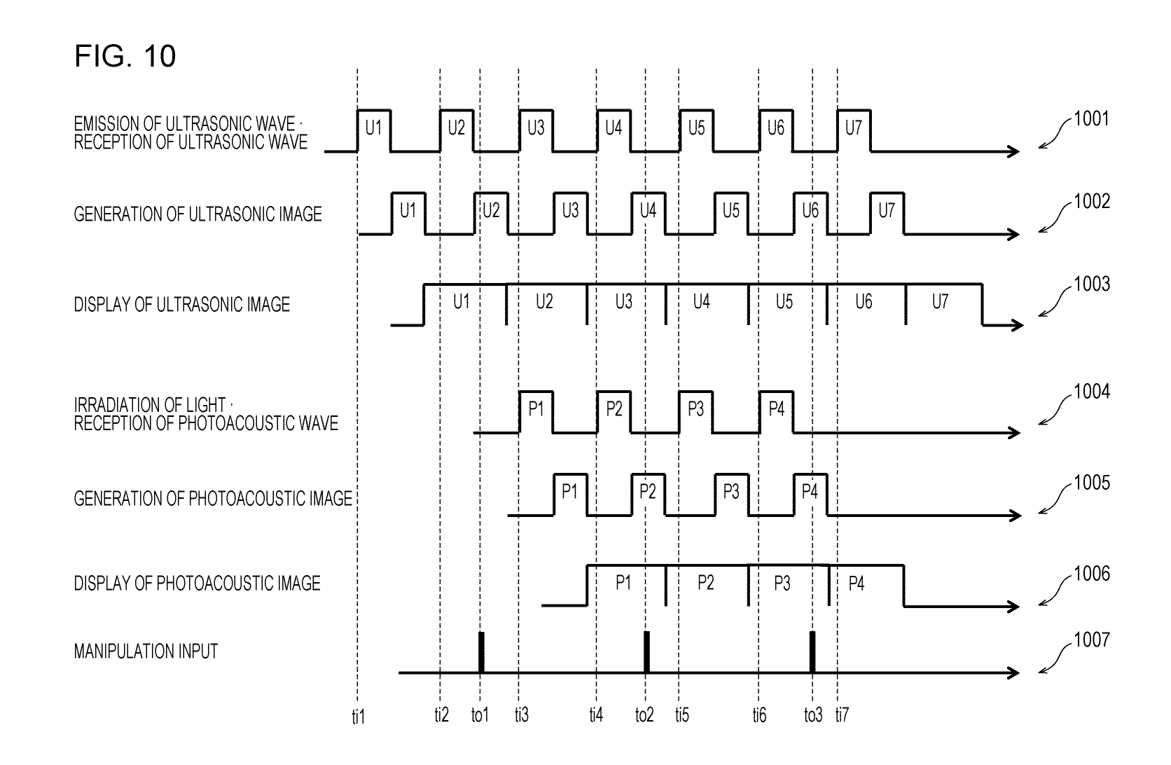

FIG. 10 is a timing chart of the processes of capturing the ultrasonic image and the photoacoustic image. Diagrams 1001 to 1007 represent that time elapses in the right-hand direction of the paper. Time ti1 to ti7 and time to1 to to3 represent time at rising points and falling points in the diagrams.

The diagram 1001 represents timing that is related to obtaining of the ultrasonic signal. At each rising point, the probe 103 starts emitting the ultrasonic waves to the test object 101, appropriately converts the obtained the reflected waves into the ultrasonic signal, and transmits the ultrasonic signal to the information-processing apparatus 107. At each falling point, the imaging control unit 302 finishes receiving the ultrasonic signal. Each of U1 to U7 represents a frame that is related to the corresponding ultrasonic image. In the frames U1 to U7, emission of the ultrasonic waves to the test object is started at the time ti1 to ti7.

The diagram 1002 represents timing that is related to obtaining of the ultrasonic image. At each raising portion, the image-processing unit 303 starts generating the ultrasonic image. At each falling point, the image-processing unit 303 finishes generating the ultrasonic image, and the information-processing apparatus 107 captures the ultrasonic image.

The diagram 1003 represents timing that is related to display of the ultrasonic image. When the ultrasonic image has been captured, the ultrasonic image can be displayed. The display control unit 306 starts displaying the frame U1 and starts displaying the frames U2 to U7 in order at a predetermined rate.

The diagram 1004 represents timing that is related to obtaining of the photoacoustic signal. At each raising point, the probe 103 starts irradiating the test object 101 with light, and the photoacoustic wave that is obtained is transmitted appropriately as the photoacoustic signal to the information-processing apparatus 107. At each falling point, the imaging control unit 302 finishes receiving the photoacoustic signal. Each of P1 to P4 represents a frame that is related to the corresponding photoacoustic image. In the frames P1 to P4, irradiation of the test object with light is started at time ti3 to ti6.

The diagram 1005 represents timing that is related to obtaining of the photoacoustic image. At each raising point, the image-processing unit 303 starts generating the photoacoustic image. At each falling point, the image-processing unit 303 finishes generating the photoacoustic image, and the information-processing apparatus 107 captures the photoacoustic image.

The diagram 1006 represents timing that is related to display of the photoacoustic image. When the photoacoustic image has been captured, the photoacoustic image can be displayed. The display control unit 306 starts displaying the frame P1 and starts displaying the frames P2 to P4 in order at a predetermined rate.

The diagram 1007 represents timing of the manipulation input of the user. At time to1 to to3, the instruction to start the photoacoustic imaging, the instruction to start the still image imaging, and the instruction to finish the photoacoustic imaging are inputted.

The step S402 of the ultrasonic imaging corresponds to the frames U1 to U4 and the frames U6 to U7 in the diagrams 1001, 1002, and 1003. The step S410 of the still image imaging corresponds to the frame U5. The step S406 of the photoacoustic imaging corresponds to the frames P1 to P2 and the frame P4 in the diagrams 1004, 1005, and 1006. The step S410 of the still image imaging corresponds to the frame P3.

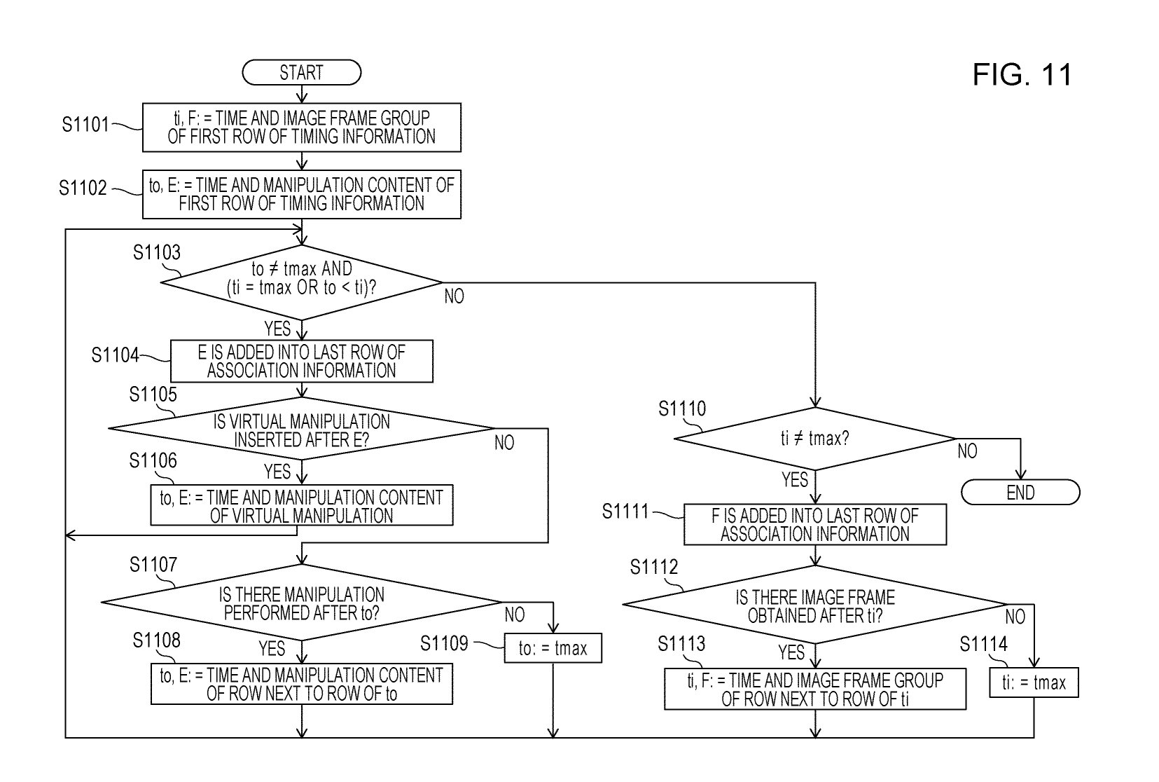

FIG. 11 is a flowchart illustrating an example of a series of processes of obtaining the association information 508 by the output control unit 304. The processes described below are performed mainly by the CPU 201 or the GPU unless otherwise particularly described.

At a step S1101, the output control unit 304 sets a temporary variable ti, which represents time at which an image is captured, at time of the first row of the timing information 506 and sets a temporary variable F, which represents an image frame group, at the image frame group of the first row of the timing information 506.

At a step S1102, the output control unit 304 sets a temporary variable to, which represents the time of the manipulation input, at time of the first row of the operation information 507 and sets a temporary variable E, which represents the content of the manipulation, at the content of the manipulation of the first row of the operation information 507.

At a step S1103 to a step S1114 described below, the output control unit 304 obtains the association information 508 on the basis of the order of timing of the manipulation input of the user, which is recorded in the operation information 507, and timing with which the image is obtained, which is recorded in the timing information 506.

At the step S1103, the output control unit 304 obtains information about tmax, to, and ti. The value of tmax is a flag value for detecting the last value of time that is recorded in the timing information 506 or the operation information 507. In the case where (1) to is not equal to tmax and (2) ti is equal to tmax or to is equal to time prior to ti, the flow proceeds to a step S1104. In the case where the above relationship is not satisfied, the flow proceeds to a step S1110.

At the step S1104, the output control unit 304 adds the content of the temporary variable E into the last row of the association information 508. The output control unit 304 may convert and add the word or form of the content of the manipulation that is written in the temporary variable E into the association information 508. For example, in the case of the association information 508 illustrated in FIG. 8, the mark “#” may be added into the head of the word that represents the content of the manipulation to manifest the fact that the information is related to the manipulation input.

At a step S1105, the output control unit 304 determines whether the virtual manipulation is inserted after E on the basis of the content of the manipulation that is represented by the temporary variable E. For example, in the case where the manipulation of E is the instruction to start the still image imaging, it is determined that the manipulation to finish the still image imaging is inserted as the virtual manipulation after E. The process for which the virtual manipulation is inserted can be set by a user in advance. In the case where it is determined that the virtual manipulation is inserted at the step S1105, the flow proceeds to a step S1106. In the case where the above determination is not made, the flow proceeds to a step S1107.

At the step S1106, the output control unit 304 sets the temporary variable E at the content of the virtual manipulation, determines time of the virtual manipulation on the basis of the content of the virtual manipulation, and sets the value of to at the time. For example, in the case where the virtual manipulation is the manipulation to finish the still image imaging, time at which the still image is captured, which is set as the temporary variable ti, is used as the time of the manipulation to finish the still image imaging. In the case where the virtual manipulation represents that a certain time t has elapsed after manipulation input E′ of the user, the time of the virtual manipulation is set at the sum of time of E′ and t.

At the step S1107, the output control unit 304 obtains information about manipulation that is performed after time that is set as the temporary variable to on the basis of the operation information 507. In the case where there is the manipulation that is performed after the time that is set as the operation information to, the flow proceeds to a step S1108. In the other case where there is not the manipulation, the flow proceeds to a step S1109.

At the step S1108, the output control unit 304 reads the time and the content of the manipulation that are written in a row next to a row of the time that is set as the temporary variable to, sets the temporary variable to at the time, and sets the temporary variable E at the content of the manipulation, on the basis of the operation information 507. Subsequently, the flow proceeds to the step S1103.

At the step S1109, the output control unit 304 sets tmax at the value of the temporary variable to. The value of tmax is a flag value for detecting the last value of the time that is recorded in the operation information 507.

At the step S1110, the output control unit 304 obtains the value that is set as ti. In the case where ti is not equal to tmax, the flow proceeds to a step S1111. In the case where ti is equal to tmax, the processes illustrated in FIG. 11 are finished.

At the step S1111, the output control unit 304 adds the image frame group that is held in the temporary variable F into the last row of the association information 508. For example, in the case where the temporary variable F has a set of the frame Um of the ultrasonic image and the frame Pn of the photoacoustic image, “(Um, Pn)” is added in the last row of the association information 508.

At a step S1112, the output control unit 304 obtains information about an image frame that is obtained after the time that is set as the temporary variable ti on the basis of the timing information 506. In the case where there is the image frame that is obtained after the time that is set as ti, the flow proceeds to a step S1113. In the other case where there is not the image frame, the flow proceeds to a step S1114.

At the step S1113, the output control unit 304 reads the time and the image frame group that are written in a row next to a row of the time that is set as the temporary variable ti, sets the temporary variable ti at the time, and sets the temporary variable F at the image frame group, on the basis of the timing information 506. Subsequently, the flow proceeds to the step S1103.

At the step S1114, the output control unit 304 sets tmax at the temporary variable ti. The value of tmax is a flag value for detecting the last value of the time that is recorded in the timing information 506. Subsequently, the flow proceeds to the step S1103.

The timing with which the ultrasonic image and the photoacoustic image are captured is saved as the timing information 506 in FIG. 6 at the step S403. The timing of the manipulation input, that is, the operation information is saved as the operation information 507 in FIG. 7 as the step S403. At the S403 to the step S411, the processes are performed in accordance with the flow illustrated in FIG. 11 on the basis of the timing information 506 and the operation information 507. Consequently, the association information 508 illustrated in FIG. 8 is obtained. At the step S413, the DICOM object 901 that includes the association information 906 illustrated in FIG. 9 is transmitted to the PACS 112.

With the structure according to the first embodiment, the operation information during imaging is associated with the image data. When the user uses the viewer 113 to display the video that includes the ultrasonic image and the photoacoustic image, the viewer 113 can efficiently display matters that are related to the manipulation input of the user on the basis of the association information 906 that is included in the DICOM object 901. For example, the viewer 113 can readily identify a frame section that is obtained together with data of the photoacoustic image in a continuous ultrasonic image frame group by referring the association information 508 that includes the operation information. The user can specify a specific point of time or section by providing the manipulation input for the operation information that is displayed on the user interface of the viewer 113. The viewer 113 displays the ultrasonic image or the photoacoustic image at a specific point of time or section on the user interface. This allows a doctor to efficiently give diagnosis. Specifically, for example, in the case where an instruction to display the superimposed image of the ultrasonic image and the photoacoustic image is received from the doctor, the viewer 113 reads time to1 and time to3 that are included in the association information 508 and obtains and displays the ultrasonic image and photoacoustic image during a period from time to1 to time to3. For example, in the case where the association information 508 includes time to1 at which imaging of the video of the photoacoustic image is started and time to3 at which the imaging is finished, the viewer 113 can identify the frame of the photoacoustic image in which the video of the photoacoustic image starts and the frame of the photoacoustic image in which the video ends. In particular, in the case where various techniques are used during a series of inspections, for example, in the case where the still image of the photoacoustic image and the still image of the ultrasonic image are imaged and the video is imaged, it is difficult to observe the images with attention paid to a specific technique merely by using information about the obtained frames. In the case where a piece of video data includes only a series of manipulations of the user and the obtained image data, it is necessary for the user to check the video data from the first frame in order to cause the viewer 113 to display the image data with the timing with which a desired manipulation is performed. With the structure according to the first embodiment, the doctor can efficiently give diagnosis. The processes according to the first embodiment enable the viewer 113 to display the image that the doctor intends to see with certainty.

### Second Embodiment

In an example described according to a second embodiment, sections between the manipulation inputs and the timing with which the image is captured are associated with each other on the basis of the timing information and the operation information.

The structure of the inspection system 102 that includes the information-processing apparatus 107 according to the second embodiment, the hardware configuration of the information-processing apparatus 107, and the functional configuration of the information-processing apparatus 107 are the same as those illustrated in FIG. 1, FIG. 2, and FIG. 3. The above description is referred for common components, and a detailed description thereof is omitted.

According to the second embodiment, the output control unit 304 saves the save data 501 illustrated in FIG. 5 in the storage device 204 at the step S403 illustrated in FIG. 4. A relationship between the sections between the manipulation inputs and the timing with which the image is captured is recorded in the association information 508 that is included in the supplementary information 502.

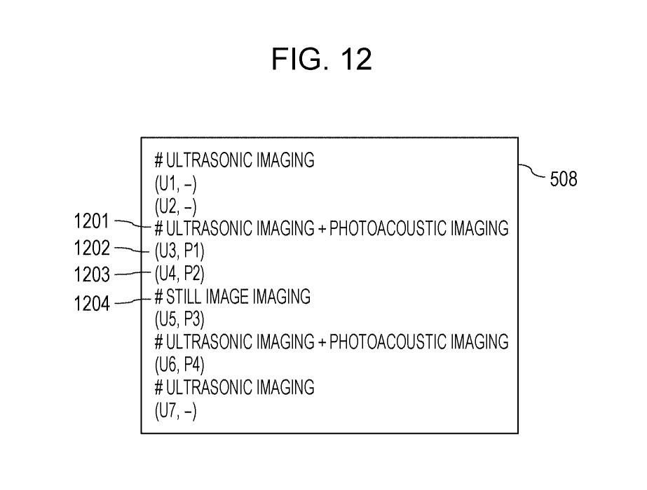

FIG. 12 illustrates an example of the association information 508. The rows that begin with the mark “#” represent the content of the manipulation inputs of the user and start of the sections (referred to below as manipulation sections) in which specific processes are performed in response to the manipulation inputs. The manipulation sections are recorded in the association information 508 in the time-series order. Each manipulation section is changed to another manipulation section with the corresponding manipulation input of the user acting as a trigger. For example, a row 1201 corresponds to the manipulation section in which the ultrasonic imaging and the photoacoustic imaging are performed, and a row 1204 corresponds to the manipulation section in which the still image imaging is performed. This is an example in which the manipulation section that is represented by the row 1201 is changed to the manipulation section that is represented by the row 1204 with the manipulation input to start the still image imaging illustrated by a row 702 in FIG. 7 acting as a trigger. After the manipulation sections, the identifier of each image that is captured between the manipulation sections is recorded in the time-series order. For example, (U3, P1) in a row 1202 represents that the frame U3 of the ultrasonic image and the frame P1 of the photoacoustic image are simultaneously obtained in the manipulation section represented by the row 1201. In addition, (U4, P2) in a row 1203 represents that the frame U4 of the ultrasonic image and the frame P2 of the photoacoustic image are substantially simultaneously obtained after U3 and P1 are obtained in the manipulation section in the row 1201.

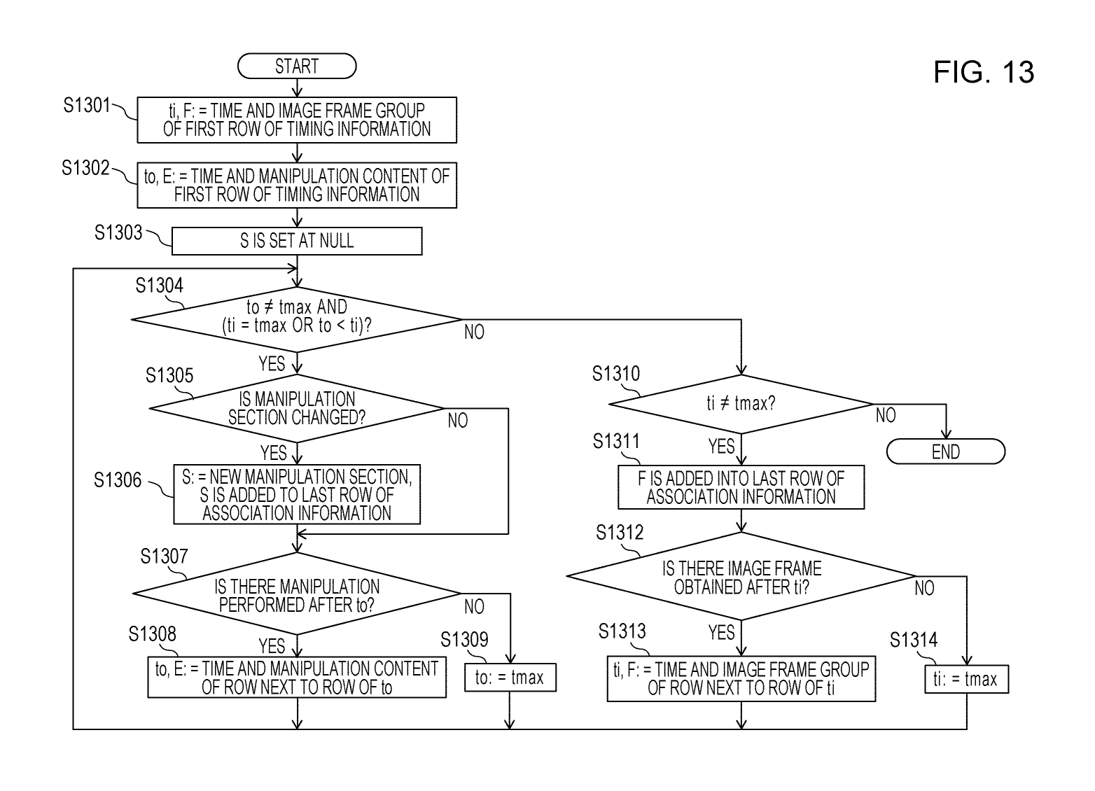

FIG. 13 is a flowchart illustrating an example of a series of processes of obtaining the association information 508 by the output control unit 304. The processes described below are performed mainly by the CPU 201 or the GPU unless otherwise particularly described.

At a step S1301, the output control unit 304 sets the temporary variable ti, which represents time at which an image is captured, at time of the first row of the timing information 506 and sets the temporary variable F, which represents an image frame group, at the image frame group of the first row of the timing information 506.

At a step S1302, the output control unit 304 sets the temporary variable to, which represents time of a manipulation instruction, at time of the first row of the operation information 507 and sets the temporary variable E, which represents the content of the manipulation, at the content of the manipulation of the first row of the operation information 507.

At a step S1303, the output control unit 304 sets a temporary variable S, which represents the manipulation sections, at NULL. In each manipulation section, processes that are related to imaging are successively performed. An example thereof is the section in which the ultrasonic imaging is performed.

At a step S1304 to a step S1314 described below, the output control unit 304 obtains the association information 508 on the basis of the order of the timing with which the image is captured and the manipulation sections between the manipulation inputs.

At the step S1304, the output control unit 304 obtains information about tmax, to, and ti. The value of tmax is a flag value for detecting the last value of the time that is recorded in the timing information 506 or the operation information 507. In the case where (1) to is not equal to tmax and (2) ti is equal to tmax or to is equal to time prior to ti, the flow proceeds to a step S1305. In the case where the above relationship is not satisfied, the flow proceeds to a step S1310.

At the step S1305, the output control unit 304 determines whether the content of the temporary variable S is changed on the basis of the content of the temporary variable E and the content of the temporary variable S. For example, in the case where S is set at NULL, E corresponds to the content of the first row of the operation information. In this case, the output control unit 304 determines that the content of S is changed to the content of E. For example, E corresponds to the manipulation to start the ultrasonic imaging, and the output control unit 304 determines that S is changed to the manipulation section in which the ultrasonic imaging is performed. In the case where S corresponds to the manipulation section in which the ultrasonic imaging is performed, and the manipulation of E corresponds to the manipulation input to start the photoacoustic imaging, the output control unit 304 determines that the manipulation section is changed to the manipulation section in which the ultrasonic imaging and the photoacoustic imaging are performed. The user can set a relationship between various conditions related to the change in the manipulation section and the content of each manipulation input in advance. In the case where it is determined that the manipulation section is changed, the flow proceeds to a step S1306. In the case where the manipulation section is not changed, the flow proceeds to a step S1307.

At the step S1306, the output control unit 304 changes the value of the temporary variable S to a new manipulation section and adds the content of S into the last row of the association information 508.

The processes at the step S1307 to the step S1314 are the same as the processes at the step S1107 to the step S1114 illustrated in FIG. 11. The above description is referred for the processes, and a detailed description thereof is omitted.

The timing with which the ultrasonic image and the photoacoustic image are captured is saved as the timing information 506 in FIG. 6 at the step S403. The timing of each manipulation input, that is, the operation information is saved as the operation information 507 in FIG. 7 at the step S403. FIG. 7 illustrates an extract of the operation information 507. In an example described below, the first row represents the manipulation to start the ultrasonic imaging, and the last row represents the manipulation to finish the ultrasonic imaging. At the step S403 to the step S411, processes are performed in accordance with the flow illustrated in FIG. 13 on the basis of the timing information 506 and the operation information 507. Consequently, the association information 508 illustrated in FIG. 12 is obtained. At the S413, the DICOM object 901 that includes the association information 906 illustrated in FIG. 9 is transmitted to the PACS 112.

With the structure according to the second embodiment, the timing of each manipulation input and the timing with which the image is captured are associated with each other. When the user uses the viewer 113 to display the video that includes the ultrasonic image and the photoacoustic image, the viewer 113 can efficiently display matters that are related to the manipulation input of the user on the basis of the association information 906 that is included in the DICOM object 901. For example, the viewer 113 can readily identify the frame section that is obtained together with data of the photoacoustic image in the continuous ultrasonic image frame group. The viewer 113 provides the frame sections that are obtained together with the data of the photoacoustic images in the ultrasonic image frame group on the user interface. The user can specify a desired frame section from the provided frame sections. The viewer 113 displays the frame section that is specified by the user on the user interface. In the case where a piece of video data includes only a series of manipulations of the user and the obtained image data, it is necessary for the user to check the video data from the first frame in order to cause the viewer 113 to display the image data with the timing with which a desired manipulation is performed. With the structure according to the second embodiment, a doctor can efficiently give diagnosis.

Modification

In the examples described according to the above embodiments, the user inputs the instruction to save the image as illustrated in FIG. 4. The present invention, however, is not limited thereto. For example, all of the images that are captured during each inspection may be saved, and the processes at the step S402 and the step S411 may not be performed.

According to the above embodiments, the ultrasonic images and the photoacoustic images are captured during a series of the inspections, and an association is established. The information-processing apparatus 107 may establish the association by using the display control unit 306. The display control unit 306 causes the display unit 109 to display the video or the still image that includes the ultrasonic image or the photoacoustic image. The display control unit 306 may cause the display unit 109 to display the superimposed image that is obtained by superimposing the ultrasonic image and the photoacoustic image that are associated with each other. In the case where the still image is imaged while the video is imaged, the display control unit 306 may identify the frame in which the still image is captured while the video is played and may cause the display unit 109 to display such that the still image is perceivable. In another example, the display control unit 306 may set the time of display of the frame that corresponds to the still image at time longer than the frame rate and may cause the display unit 109 to display.

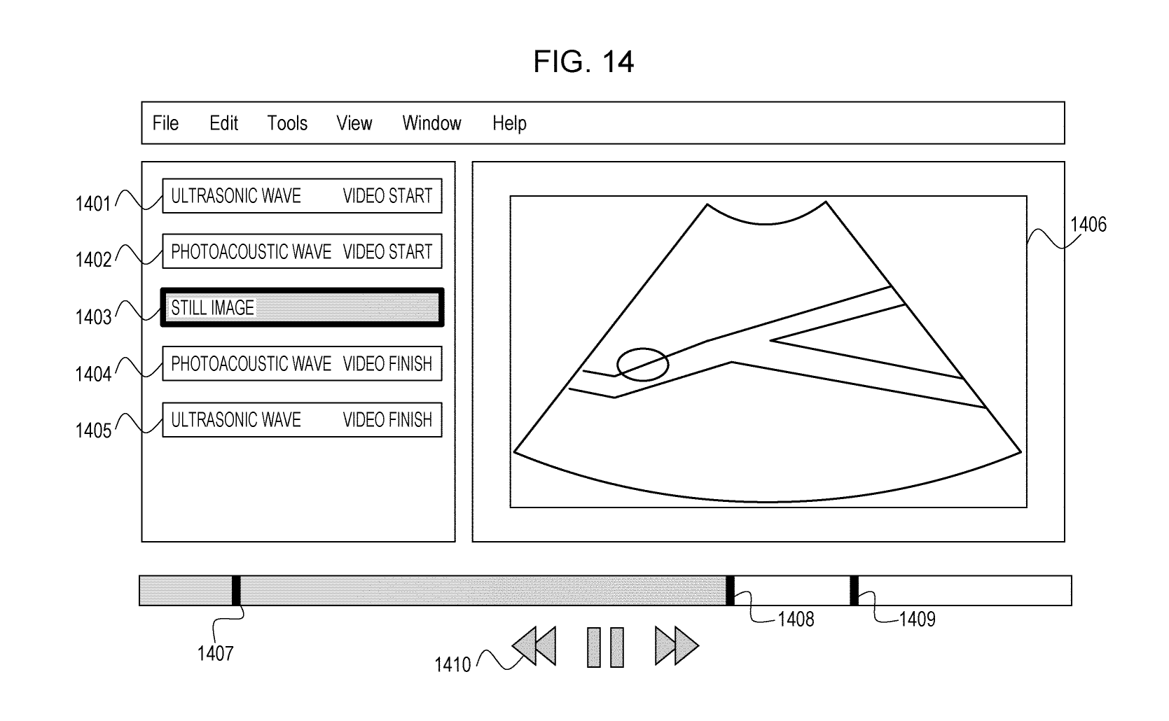

FIG. 14 illustrates an example of a screen of a display device (not illustrated) that displays a medical image on the basis of information that is obtained by the information-processing apparatus according to one of the embodiments of the present invention. An example of the display device (not illustrated) is a computer and is connected to the information-processing apparatus 107 so as to be able to communicate with the information-processing apparatus 107. The display device (not illustrated) may be an image-inspecting device, may be a computer that is used by a doctor to observe a medical image, or may be a diagnosis assistance device. The display device (not illustrated) obtains the DICOM object from the information-processing apparatus 107. The display device (not illustrated) obtains, from the PACS 112, the DICOM object that is transmitted from the information-processing apparatus 107 to the PACS 112 and that is saved.

In an example described below, the display device (not illustrated) obtains the DICOM object 901 illustrated in FIG. 9. The display device (not illustrated) reads the supplementary information 902 and the image data 903 from the DICOM object 901. The display device (not illustrated) displays the image data 903 such that the supplementary information 902 can be referred.

The display device (not illustrated) displays a medical image 1406. The image data 903 is video data, and a progress bar for the video and buttons 1410 for the manipulation inputs that are related to playback are displayed. Buttons 1401 to 1405 are associated with the association information 906 that is included in the supplementary information 902. FIG. 8 illustrates the content of the association information 906. The button 1401 corresponds to the content of the row 801. The button 1402 corresponds to the content of the row 804. The button 1403 corresponds to the content of the row 805. The button 1404 corresponds to the content of a row 807. The button 1405 corresponds to the content of a row 808.

The display device (not illustrated) provides a marker function to make it easy for a doctor (user) to observe the medical image that related to the association information 906. The button 1401 corresponds to the start position of the video. The button 1402 corresponds to a marker 1407. The button 1403 corresponds to a marker 1408. The button 1404 corresponds to a marker 1409. The button 1405 corresponds to the end position of the video. When the user provides a manipulation input to push any one of the button 1401 to the button 1405, the display device (not illustrated) displays the corresponding medical image, that is, the medical image at the corresponding position of the video. FIG. 14 illustrates an example in which the button 1403 is pushed. This is the still image that is imaged while the ultrasonic video and the photoacoustic video are imaged. The playback of the video skips to the position of the marker 1408, and the superimposed image of the ultrasonic image that is represented by the frame U5 and the photoacoustic image that is represented by the frame P3 illustrated in FIG. 8 are displayed as the medical image 1406.

On the basis of the DICOM object that is obtained by the information-processing apparatus according to each embodiment of the present invention, the medical image with the timing with which a shooter who takes the medical image manipulates can be readily displayed.

The present invention can also be carried out in a manner in which the system or the apparatus is provided with a program for performing one or more functions according to the above embodiments via a network or a storage medium, and one or more processors of a computer of the system or the apparatus read and execute the program. The present invention can also be carried out by a circuit (for example, an ASIC) for performing one or more functions.

The information-processing apparatus according to each embodiment described above may be a single apparatus, or a plurality of apparatuses may be combined so as to be able to communicate with each other to perform the above processes. These are included in the embodiments of the present invention. The above processes may be performed by a common server apparatus or a server group. It is not necessary for a plurality of apparatuses that achieve the information-processing apparatus and the information-processing system to be installed in the same facility or the same country provided that the apparatuses can communicate at a predetermined communication rate.

The embodiments of the present invention include an embodiment in which the system or the apparatus is provided with a software program that performs the functions according to the above embodiments, and the computer of the system or the apparatus reads and executes codes of the provided program.

Accordingly, the program codes that are installed in the computer to perform the processes according to the embodiments by the computer are included in the embodiments of the present invention. The functions according to the above embodiments can be performed in a manner in which an OS that acts on the computer, for example, performs a part or all of actual processing on the basis of instructions that are included in the program that the computer reads.

An appropriate combination of the above embodiments is also included in the embodiments of the present invention.

The present invention is not limited to the above embodiments. Various modifications and alterations can be made without departing form the spirit and scope of the present invention. Accordingly, the following claims are attached to publish the scope of the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An information-processing apparatus comprising:
an image-capturing unit that captures either or both of an ultrasonic image and a photoacoustic image that are imaged by an imaging device;
an information-obtaining unit that obtains operation information about manipulation of the imaging device for instructing an imaging method and time of the manipulation regarding either or both of the ultrasonic image and the photoacoustic image and that obtains time information about either or both of time at which the ultrasonic image is captured and time at which the photoacoustic image is captured; and
an output unit that outputs, to an external device, the operation information, the time information, and either or both of the ultrasonic image and the photoacoustic image that are associated with each other.

2. The information-processing apparatus according to claim 1, wherein the operation information includes information about start of the imaging method and information about end of the imaging method.

3. The information-processing apparatus according to claim 1, wherein the operation information includes information about automatic operation based on the manipulation of a user and time of the automatic operation.

4. The information-processing apparatus according to according to claim 1, wherein the output unit outputs, to the external device, supplementary information that is perceivably associated with an order of time at which either or both the ultrasonic image and the photoacoustic image are captured and the time of the manipulation.

5. The information-processing apparatus according to claim 1, further comprising: an image-capturing unit that captures the photoacoustic image on the basis of a photoacoustic signal and captures the ultrasonic image on the basis of an ultrasonic signal.

6. The information-processing apparatus according to claim 1, wherein the information-obtaining unit also obtains timing information about timing with which an ultrasonic signal and a photoacoustic signal are obtained, and
wherein the output unit outputs, to the external device, association information about a relationship between the ultrasonic image and the photoacoustic image, the association information being obtained on the basis of the timing information and further associated.

7. The information-processing apparatus according to claim 1, wherein the imaging method is related to either or both of imaging of the ultrasonic image or the photoacoustic image that is included in a video and imaging of the ultrasonic image or the photoacoustic image that is included in a still image.

8. An information-processing apparatus comprising:
an obtaining unit that obtains supplementary information that includes operation information about manipulation of an imaging device for instructing an imaging method and time of the manipulation and an object that includes image data of either or both of an ultrasonic image and a photoacoustic image that are associated with the operation information; and
a display-controlling unit that reads the operation information and that causes a display unit to display the image data that is obtained by the manipulation.

9. The information-processing apparatus according to claim 8, wherein the display-controlling unit causes the display unit to display information about the operation information,
wherein the information-processing apparatus further includes a reception unit that receives a manipulation input of a user in response to the displayed information about the operation information, and
wherein the display-controlling unit causes the display unit to display the image data that is obtained by the manipulation that corresponds to the information about the operation information and that is received.

10. An information-processing system comprising:
an image-capturing unit that captures either or both of an ultrasonic image and a photoacoustic image that are imaged by an imaging device;
an information-obtaining unit that obtains operation information about manipulation of the imaging device for instructing an imaging method and time of the manipulation regarding either or both of the ultrasonic image and the photoacoustic image and that obtains time information about either or both of time at which the ultrasonic image is captured and time at which the photoacoustic image is captured; and
an output unit that outputs, to an external device, the operation information, the time information, and either or both of the ultrasonic image and the photoacoustic image that are associated with each other.

11. An information-processing system comprising:
an obtaining unit that obtains supplementary information that includes operation information about manipulation of an imaging device for instructing an imaging method and time of the manipulation and an object that includes image data of either or both of an ultrasonic image and a photoacoustic image that are associated with the operation information; and
a display-controlling unit that reads the operation information and that causes a display unit to display the image data that is obtained by the manipulation.

12. A method for processing information, the method comprising:
an image-capturing step of capturing either or both of an ultrasonic image and a photoacoustic image that are imaged by an imaging device;
an information-obtaining step of obtaining operation information about manipulation of the imaging device for instructing an imaging method and time of the manipulation regarding either or both of the ultrasonic image and the photoacoustic image and obtaining time information about either or both of time at which the ultrasonic image is captured and time at which the photoacoustic image is captured; and
an output step of outputting, to an external device, the operation information, the time information, and either or both of the ultrasonic image and the photoacoustic image that are associated with each other.

13. A method for processing information, the method comprising:
a first step of obtaining operation information about manipulation for imaging either or both of an ultrasonic image and a photoacoustic image by an imaging device and time of the manipulation;
a second step of obtaining time information about either or both of time at which the ultrasonic image is captured and time at which the photoacoustic image is captured;
a third step of obtaining supplementary information on the basis of the operation information and the time information; and
a fourth step of causing a display unit to perceivably display a relationship between the manipulation and either or both of the captured ultrasonic image and photoacoustic image on the basis of the supplementary information.

14. A method for processing information, the method comprising:
a first step of obtaining operation information about manipulation for imaging either or both of an ultrasonic image and a photoacoustic image by an imaging device and time of the manipulation;
a second step of obtaining time information about either or both of time at which the ultrasonic image is captured and time at which the photoacoustic image is captured;
a third step of causing a display unit to display the information about the manipulation;
a fourth step of receiving a specification of a user in response to the information about the manipulation; and
a fifth step of causing the display unit to display either or both of the ultrasonic image and the photoacoustic image on the basis of time of the manipulation of the specification and the time information.

15. A non-transitory computer-readable medium storing a program for causing a computer to execute the method according to 12 for processing information.

16. A non-transitory computer-readable medium storing a program for causing a computer to execute the method according to 13 for processing information.

17. A non-transitory computer-readable medium storing a program for causing a computer to execute the method according to 14 for processing information.

18. The information-processing apparatus according to claim 1, wherein the output unit outputs, to the external device, a DICOM object that includes the operation information, the time information, and either or both of the ultrasonic image and the photoacoustic image.

19. The information-processing apparatus according to claim 8, wherein the display-controlling unit causes the display unit to display the operation information, time that is related to the operation information, and the image data that are arranged.
